(19) United States
(12) Patent Application Publication
(10) Pub. No.: US 2023/0000682 A1
(43) Pub. Date: Jan. 5, 2023

(54) SURGICAL INSTRUMENT

(71) Applicant: ART, LIMITED, Grand Cayman (KY)

(72) Inventors: Ravi Nallakrishnan, Willowbrook, IL (US); Takayuki Akahoshi, Tokyo (JP)

(21) Appl. No.: 17/782,084

(22) PCT Filed: Jan. 4, 2021
(86) PCT No.: PCT/US21/12030

§ 371 Date: Jun. 2, 2022

Related U.S. Application Data
(60) Provisional application No. 62/957,849, filed on Jan. 7, 2020.

(51) Int. Cl. A61F 9/013

(52) U.S. Cl. CPC A61F 9/0133

(57) ABSTRACT

A surgical blade (10) for being combined with an elongate handle (50) to form a cutting instrument for ophthalmic surgery includes a proximal portion (12) for being attached to the handle (12), an operative, distal portion (14), and a central portion (20) extending therebetween. The central portion (20) having a trapezoidal configuration that tapers radially inwardly from the proximal portion (12) to the distal portion and having a pair of sharpened lateral edges (21, 22). The distal portion (14) includes a first tapered section (23) defining a pair of sharpened lateral edges (24, 25) and extending from the central portion (20) to a substantially straight section (26) having a pair of sharpened lateral edges (27, 28). The substantially straight section (26) extends from the first tapered section (23) to a second tapered section (30) having a pair of sharpened lateral edges (32, 33) and terminating at a pointed tip (36).

### FIELD OF THE INVENTION

The present invention relates to a surgical instrument for performing ophthalmological procedures for treatment of eye diseases, such as cataract, and more particularly to a knife or blade for being incorporated into a knife for making corneal incisions.

### BACKGROUND OF THE INVENTION

Phacoemulsification has come to be a technique of choice for the removal of damaged or diseased natural lenses from the eye. Commonly, such surgery is called for when a patient develops cataracts, a condition in which a portion of the eye lens becomes hard and cloudy or opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. One of the most significant advantages of the use of phacoemulsification is that the apparatus itself is small and can fit through a relatively small incision, e.g., 0.5-3.0 mm, resulting in less fluid leakage from the eye capsule and shorter patient recovery times. Multiple incisions of varying sizes are typically made in the cornea to accommodate the different surgical instruments used in the surgery, such as forceps, irrigation/aspiration needle, nucleus chopper, intraocular lens (hereinafter “IOL”) injector, etc. In some surgeries, a larger incision or main port of about 1.8 mm is made to accommodate the implantation of a folded intraocular lens, which will expand and unfold in the eye, while one or two side ports or incisions of about 0.5 mm are made to accommodate irrigation sources or sustainers.

It is desirable to limit the trauma to the eye, by reducing the size of the incision, to minimize the risk of long-term damage to eye tissue and complications arising from surgery. Minimizing the size of the incision also helps to maintain intraocular pressure.

Known designs for ophthalmological knives to make corneal incisions generally have a beveled, natural diamond material blade with a triangular or trapezoidal shape to make a path through the cornea and into the anterior chamber of the eye. U.S. Pat. No. 6,547,802, hereby incorporated by reference in its entirety, discloses a surgical knife for making incisions in the cornea. Additional ophthalmological knives are sold by American Surgical Instruments Corporation having offices located at 26 Plaza Drive, Westmont, Ill. 60559, U.S.A. under the product serial nos. AE-8190, AE-8192, and AE-8121.

At present there remains a need in the art for the development of simple, inexpensive, and durable instruments useable to perform the procedure of creating one or more ports or incisions in the eye to minimize the likelihood of developing complications during the healing process.

The present invention is directed to an improved construction for an ophthalmic surgical blade, which may be incorporated into a knife, which addresses one or more of the above-discussed problems.

### SUMMARY OF THE INVENTION

In accordance with one form of the present invention, a surgical blade is disclosed for being combined with an elongate handle to form a cutting instrument for ophthalmic surgery includes a proximal portion for being attached to an elongate handle, an operative, distal portion, and a central portion extending therebetween. The central portion having a trapezoidal configuration that tapers radially inwardly from the proximal portion to the distal portion and having a pair of sharpened lateral edges. The distal portion includes a first tapered section defining a pair of sharpened lateral edges and extending from the central portion to a substantially straight section having a pair of sharpened lateral edges. The substantially straight section extends from the first tapered section to a second tapered section having a pair of sharpened lateral edges and terminating at a pointed tip.

In another aspect of the present invention, the first tapered section defines an internal angle of between about 30 degrees and about 70 degrees, and more preferably about 50 degrees between its pair of sharpened lateral edges.

In another aspect of the present invention, the second tapered section defines an internal angle of between about 30 degrees and about 70 degrees, and more preferably about 50 degrees between its pair of sharpened lateral edges.

In yet another aspect of the present invention, the substantially straight section defines a substantially constant width between its pair of sharpened lateral edges.

In one aspect of the present invention, a total length of the central portion is between about 1.5 and 1.7 times greater than a total length of the distal portion, as measured along the central longitudinal axis of the blade.

In still another form of the present invention, the distal portion defines a total length, taken along the central longitudinal axis, and the first tapered section has a length of about 62.5% of the total length, the substantially straight section has a length of about 12.5% of the total length, and the second tapered section has a length of about 25% of the total length.

In another aspect of the present invention, the first tapered section has a maximum width of between about 3 and 3.5 times greater than a maximum width of the second tapered section, taken in the radial direction relative to the central longitudinal axis.

According to another aspect of the present invention, the sharpened lateral edges are beveled between top and bottom sides of the blade.

In accordance with another form of the present invention, a surgical blade is disclosed for being combined with an elongate handle to form a cutting instrument for ophthalmic surgery includes a proximal portion for being attached to an elongate handle, an operative, distal portion, and a central portion extending therebetween. The central portion has a trapezoidal configuration that tapers radially inwardly from the proximal portion to the distal portion and having a pair of sharpened lateral edges. The distal portion tapers to a pointed tip and defines a pair of sharpened lateral edges defining an internal angle of about 50 degrees.

In another form of the present invention, the blade has a thickness between top and opposite bottom sides of between about 100 and 150 micrometers.

According to another aspect of the present invention, the blade is formed from one of natural diamond, black diamond (carbon), sapphire, ruby, or stainless steel.

According to another aspect of the present invention, the blade is in combination with an elongate handle, wherein the combination of the blade and the handle form a cutting instrument.

In still another form of the present invention, the proximal portion includes a bend leading toward the central portion and the distal portion, the bend defining a pair of lateral sharpened edges.

### BRIEF DESCRIPTION OF THE DRAWINGS

In the accompanying drawings forming part of the specification, in which like numerals are employed to designate like parts throughout the same,

FIG. 1 is a greatly enlarged, top elevation view of a first embodiment of a surgical instrument according to the present invention, and FIG. 1 shows a blade for being attached to a handle (not illustrated), the blade and handle together comprising a knife;

FIG. 2 is a greatly enlarged, top elevation view of a second embodiment of a surgical instrument according to the present invention, and FIG. 2 shows a blade for being attached to a handle (not illustrated), the blade and handle together comprising a knife;

FIG. 3 is a greatly enlarged, top elevation view of a third embodiment of a surgical instrument according to the present invention, and FIG. 3 shows a blade for being attached to a handle (not illustrated), the blade and handle together comprising a knife;

FIG. 4 is a greatly enlarged, top elevation view of a fourth embodiment of a surgical instrument according to the present invention, and FIG. 4 shows a blade for being attached to a handle (not illustrated), the blade and handle together comprising a knife;

FIG. 5 is a greatly enlarged, top elevation view of a fifth embodiment of a surgical instrument according to the present invention, and FIG. 5 shows a blade for being attached to a handle (not illustrated), the blade and handle together comprising a knife;

FIG. 6 is a greatly enlarged, top elevation view of a sixth embodiment of a surgical instrument according to the present invention, and FIG. 6 shows a blade for being attached to a handle (not illustrated), the blade and handle together comprising a knife;

FIG. 7 is a greatly enlarged, fragmentary, top elevation photographic view of a seventh embodiment of a surgical instrument according to the present invention, and FIG. 7 shows a blade in combination with handle, wherein the blade is attached to a distal portion of a handle, the blade and handle together comprising a knife;

FIG. 8 is a greatly enlarged, fragmentary, left side elevation view of the portion of the instrument illustrated in FIG. 7;

FIG. 9 is a greatly enlarged, diagrammatic, cross-sectional view of the human eye; and

FIG. 10 is top elevation view of the surgical instrument of FIG. 1, and FIG. 10 shows the blade in combination with a handle to define a knife.

### DESCRIPTION OF THE PREFERRED EMBODIMENTS

While this invention is susceptible of embodiment in many different forms, this specification and the accompanying drawings disclose only specific forms as examples of the invention. The invention is not intended to be limited to only the embodiments so described, and the scope of the invention will be pointed out in the appended claims.

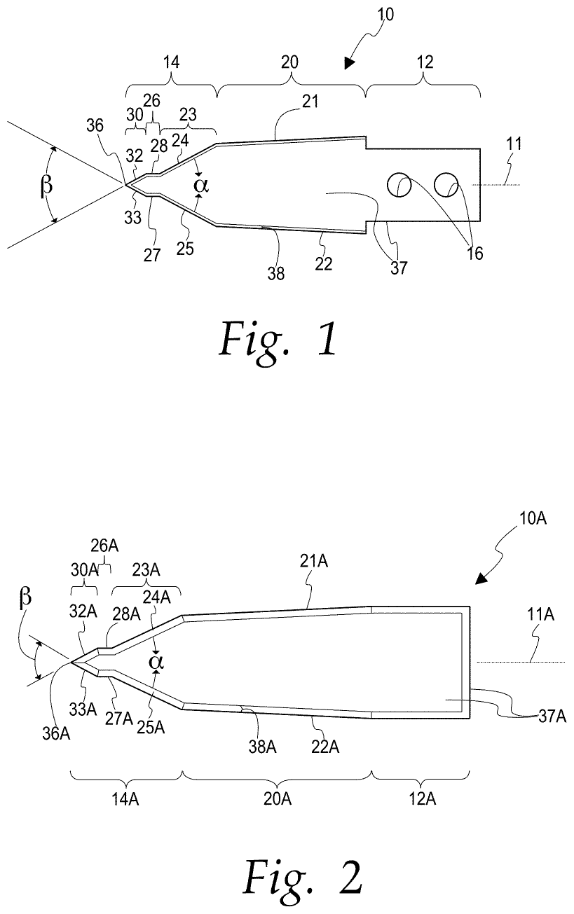

A first embodiment of a surgical instrument of the present invention is illustrated in FIG. 1, wherein only the blade 10 of the instrument is illustrated in FIG. 1. It will be understood by one of ordinary skill in the art that the blade 10 would be assembled with a handle 50 (FIG. 10) to form an instrument in the form of a knife for ophthalmic surgery. The handle of the instrument is elongated for being gripped by a user of the instrument, and can be provided with either a rounded cross-sectional configuration or with a flattened, polygonal, or irregular cross-sectional configuration. The handle has a proximal end, and a distal end to which the blade 10 is secured by either conventional or special means. The blade 10 may be sold to a user separately from the handle 10.

Referring to FIG. 1, the blade 10 defines a central longitudinal axis 11 and includes a proximal portion 12 and distal portion 14. The proximal portion 12 is configured for being attached to the distal end of a handle 50 (FIG. 10) and has a pair of circular apertures 16 for receiving mating protrusions in the handle to securely clamp and retain the blade 10 in the handle 50. Other means may be used for securing the blade 10 to the handle 50, such as friction fitting, welding, adhesive, overmolding, or forming the blade 10 as a unitary part of the handle 50 etc. A central portion 20 of the blade 10 extends between the proximal portion 12 and the distal portion 14 and has a trapezoidal configuration that tapers from the proximal portion 12 to the distal portion 14 and has a pair of sharpened lateral edges 21 and 22. In one presently preferred form of the blade 10, the central portion has a length, direction along the axis 11, of about 4 mm with a width, direction normal to the axis 11, of about 2.4 mm at the proximal portion 12 (wider end) and a width of about 2 mm at the distal portion 14 (narrower end).

Still referring to FIG. 1, the distal portion 14 of the blade 10 is further defined by discrete sections or sub-portions, which will now be discussed. A first tapered section 23 extends axially away (distally) along axis 11 from the narrower end of the central portion 20 toward a straight section 26. The first tapered section 23 defines a pair of sharpened lateral edges 24 and 25. In one presently preferred form of the blade 10, the first tapered section 23 has a length of about 1.5 mm with a width of about 2.0 mm at the central portion 20 and a width of about 0.6 mm at the straight section 26, and the lateral edges 24 and 25 define an interior angle α of about 50 degrees.

The straight section 26 of the distal portion 14 of the blade 10 extends along the central axis 11 distally away from the first tapered section 23 and has a substantially constant width between two sharpened lateral edges 27 and 28. In one presently preferred form of the blade 10, the straight section 26 has a length of about 0.3 mm with a width of about 0.6 mm.

Still referring to FIG. 1, the straight section 26 of the distal portion 14 of the blade 10 terminates at a second tapered section 30. The second tapered section 30 extends axially away (distally) along axis 11 from the straight section 26. The second tapered section 30 defines a pair of sharpened lateral edges 32 and 33 that terminate in a point or tip 36. In one presently preferred form of the blade 10, the second tapered section 30 has a length of about 0.6 mm, and the lateral edges 32 and 33 define an interior angle β of about 50 degrees. It can be seen that the lateral edges 21, 22, 24, 25, 27, 28, 32 and 33 define a bevel 38 to form cutting edges around the entire perimeter of the middle portion 20 and distal portion 14 of the blade 10. In one presently preferred form of the blade 10, the blade 10 has a nominal thickness of between 100 μm and 150 μm, and the bevel 38 is between about 15-45 degrees, and more preferably 30 degrees between the sides 37 (only the top side being visible in FIG. 1 and the understanding that the bottom side is substantially flat) of the blade 10. In some forms, the bottom side could be a mirror image of the top side and could have a matching bevel.

The blade 10 is preferably formed from black diamond (carbon), but could be formed from natural diamond, sapphire, ruby, or stainless steel. In some applications, the blade may have a coating applied to one or more of the above-discussed materials functioning as a substrate to improve wear resistance, reflectivity, etc. In one preferred form, the coating is a chemical vapor deposition “CVD” or a diamond-like deposit on the substrate. The blade 10 or coating on a substrate of the blade 10 may be formed with hydrophobic and/or hydrophilic surfaces to enhance the gliding of the blade 10. The inventors have found that artificial diamond carbon material is surprisingly suitable for this application in the blade 10 and may result in a reduction in the material cost of the blade of about 10× compared to the cost of natural diamond. The black diamond (carbon) may also greatly improve visibility of the blade within the eye during surgery when compared to blades made from natural diamond.

The inventors of the present invention have found that using the blade 10 in a knife as described above performs surprisingly well for the formation of the main port or incision in the cornea and one or more side ports or incisions. This reduces the number of knives that may be required for the ophthalmic surgery, which would reduce the number of instruments needing sterilization post-surgery. Furthermore, the ability of the user to make the main and side ports with a single instrument can improve the speed and efficiency of surgery.

The handle 50 of the knife that is assembled with the blade 10 could be a rigid elongate metal such as aluminum or titanium and may be provided with mechanisms to facilitate movement of the blade 10 along the axis 11 relative to the handle, such as a spring, slide or screw threading.

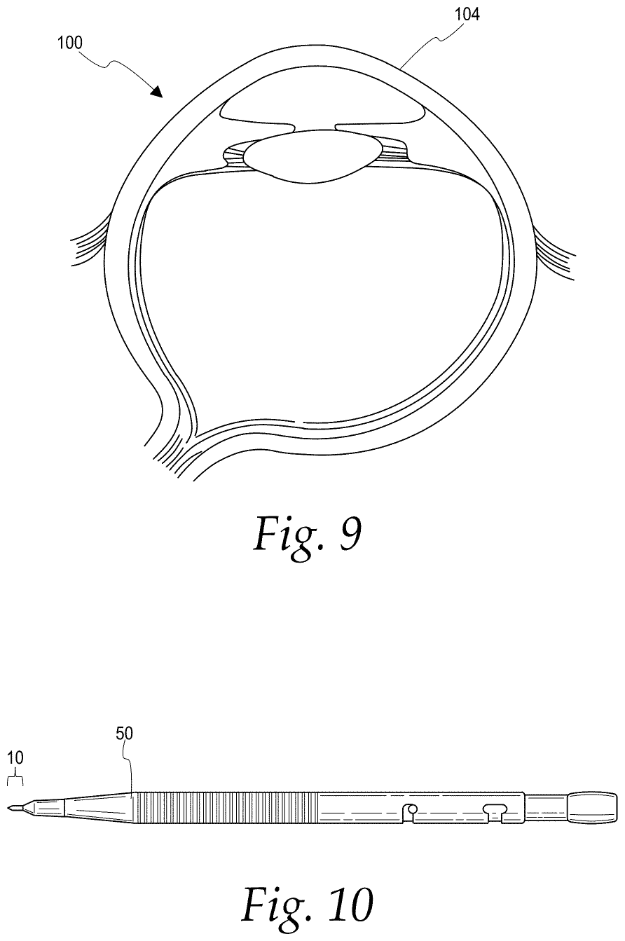

In one preferred method of operation of a knife utilizing the blade 10, the user would create at least two incisions or ports within the eye (100 in FIG. 9). The main port in the cornea (104 in FIG. 9) would be made by extending the blade 10 into the eye 100 up to, or near, the 2.0 mm wide portion of the blade 10 (near the boundary of the middle portion 20 and the distal portion 14). The side port or ports in the cornea 104 would be made by extending the blade 10 to or near 0.6 mm wide portion of the blade 10 (near the boundary of the second tapered section 30 and the straight section 26 of the distal portion 14). The main port may be used to accommodate the insertion of 2 mm or sub-2 mm micro-coaxial cataract instruments.

It is believed that the blade 10 is well suited for performing the clear corneal surgical technique to make an incision that seals itself and does not require sutures to prevent leakage of fluid from the anterior chamber of the eye.

A second embodiment of a surgical instrument of the present invention is illustrated in FIG. 2, wherein only the blade 10A of the instrument is illustrated. It will be understood by one of ordinary skill in the art that the blade 10A would be assembled with a handle (such as the handle 50 in FIG. 10) to form a knife for ophthalmic surgery. The numbered features of the second embodiment of the blade 10A illustrated in FIG. 2 are designated generally with the suffix letter “A” and are analogous to features of the first embodiment of the blade 10 that share the same number (without the suffix letter “A”). The second illustrated embodiment of the blade 10A functions identically to the blade 10 of the first illustrated embodiment. The blade 10A differs in that the proximal portion 12A of the blade 10A does not possess any apertures for mounting or assembling with a handle.

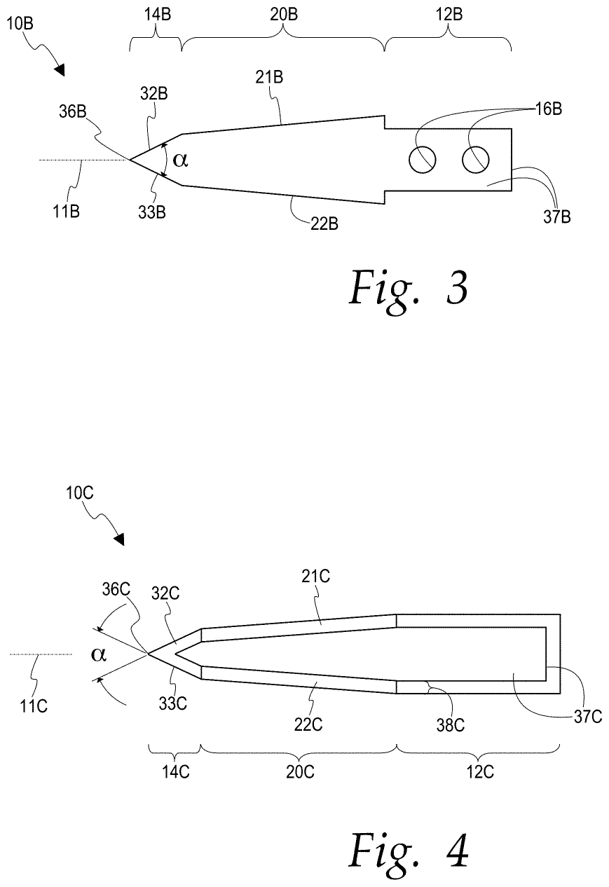

A third embodiment of a surgical instrument of the present invention is illustrated in FIG. 3, wherein only the blade 10B of the instrument is illustrated. It will be understood by one of ordinary skill in the art that the blade 10B would be assembled with a handle (such as the handle 50 in FIG. 10) to form a knife for ophthalmic surgery. The numbered features of the third embodiment of the blade 10B illustrated in FIG. 3 are designated generally with the suffix letter “B” and are analogous to features of the first embodiment of the blade 10 that share the same number (without the suffix letter “B”). The third illustrated embodiment of the blade 10B functions in a similar manner to the blade 10 of the first illustrated embodiment. The blade 10B differs in that it is utilized for making a single sized incisions (e.g., side port incisions) only and does not possess the requisite cutting features for creating incisions of multiple sizes (e.g., main port incision and side port incisions).

The blade 10B is similar in nature to the first illustrated embodiment of the instrument 10 and includes a central longitudinal axis 11B, a proximal portion 12B and distal portion 14B. The proximal portion 12B is configured for being attached to the distal end of a handle (not illustrated in FIG. 3) and has a pair of circular apertures 16B for receiving mating protrusions in the handle to securely clamp and retain the blade 10B in the handle. Other means may be used for securing the blade 10B to the handle, such as friction fitting, welding, adhesive, overmolding, etc. A central portion 20B of the blade 10B extends between the proximal portion 12B and the distal portion 14B and has a trapezoidal configuration that tapers from the proximal portion 12B to the distal portion 14B and has a pair of cutting or sharpened lateral edges 21B and 22B. In one presently preferred form of the blade 10B, the central portion 20B has a length of about 2.4 mm along the central axis 11B and a width (in the direction normal to the central axis 11B) of about 1.0 mm at the proximal portion 12B and a width of about 0.6 mm at the distal portion 14B.

Still referring to FIG. 3, the distal portion 14B of the blade 10B is tapered as it extends axially away (distally) along axis 11B from the narrower end of the central portion 20B toward a point of termination 36B. The distal portion 14B defines a pair of sharpened lateral edges 32B and 33B defining an interior angle α. In one presently preferred form of the blade 10B, the distal portion 14B of the blade 10B has a length of about 0.6 mm with a width of about 0.6 mm at the central portion 20B and then tapering to the point 36B, and the lateral edges 32B and 33B define an interior angle α of about 50 degrees. One or more of the sharpened lateral edges 21B, 22B, 32B and 33B may be beveled and may form cutting edges around the entire perimeter of the middle portion 20B and distal portion 14B of the blade 10B. In one presently preferred form of the blade 10B, the blade 10B has a nominal thickness of between 100 μm and 150 μm, between opposite sides 37B of the blade 10B (only the top side 37B being visible in FIG. 3). In some forms, the bottom side 37B could be a mirror image of the top side 37B, or it may be beveled instead of being substantially flat.

The blade 10B is preferably formed from black diamond (carbon), but could be formed from natural diamond, sapphire, ruby, or stainless steel. In some applications, the blade may have a coating to improve wear resistance, reflectivity, etc. In one preferred form, the coating is a chemical vapor deposition “CVD” or a diamond-like deposit on the substrate. The blade 10B or coating on a substrate of the blade 10B may be formed with hydrophobic and/or hydrophilic surfaces to enhance the gliding of the blade 10B.

The inventors have found that using the blade 10B in a knife as described above performs surprisingly well for the formation of the side ports or incisions in the cornea which can improve the speed and efficiency of surgery and reduce trauma to the eye and furthermore the blade 10B has a significantly reduced cost compared to diamond blades on the market without sacrificing sharpness and/or durability.

The handle of the knife that is assembled with the blade 10B could be a rigid elongate metal such as titanium and may be provided with mechanisms to facilitate movement of the blade 10B along the axis 11B relative to the handle, such as a spring, slide or screw threading.

A fourth embodiment of a surgical instrument of the present invention is illustrated in FIG. 4, wherein only the blade 10C of the instrument is illustrated. It will be understood by one of ordinary skill in the art that the blade 10C would be assembled with a handle (such as the handle 50 in FIG. 10) to form a knife for ophthalmic surgery. The numbered features of the fourth embodiment of the blade 10C illustrated in FIG. 4 are designated generally with the suffix letter “C” and are analogous to features of the third embodiment of the blade 10B that share the same number (without the suffix letter “C”). The fourth illustrated embodiment of the blade 10C functions identically to the blade 10B of the third illustrated embodiment. The blade 10C differs in that the proximal portion 12C of the blade 10C does not possess any apertures for mounting or assembling with a handle.

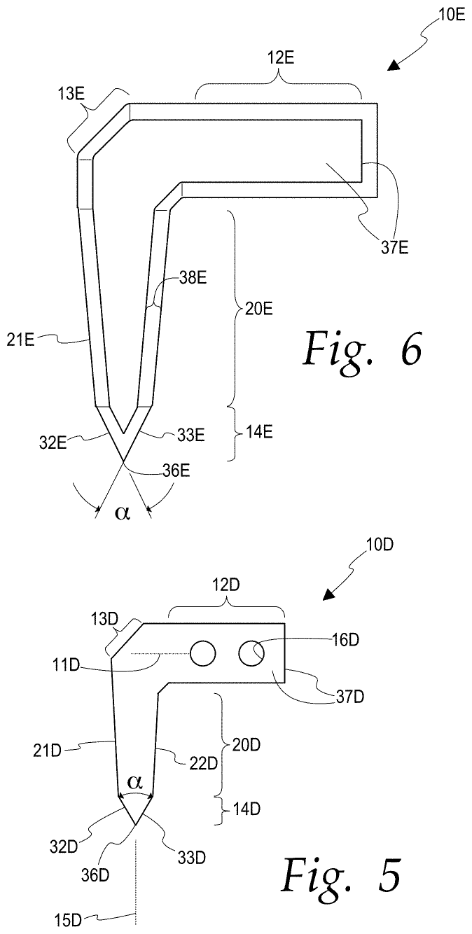

A fifth embodiment of a surgical instrument of the present invention is illustrated in FIG. 5, wherein only the blade 10D of the instrument is illustrated. It will be understood by one of ordinary skill in the art that the blade 10D would be assembled with a handle (such as the handle 50 in FIG. 10) to form a knife for ophthalmic surgery. The numbered features of the fifth embodiment of the blade 10D illustrated in FIG. 5 are designated generally with the suffix letter “D” and are analogous to features of the third embodiment of the blade 10B that share the same number (without the suffix letter “D”). The fifth illustrated embodiment of the blade 10D functions similarly to the blade 10B of the third illustrated embodiment. The blade 10D differs in that the proximal portion 12D of the blade 10D includes a bend 13D leading toward the central portion 20D and the distal portion 14D.

The blade 10D defines a longitudinal axis 11D and a transverse, horizontal axis 15D. In one presently preferred form of the blade 10D, the longitudinal axis 11D and a horizontal axis 15D are normal to one another. Preferably, the central portion 20D of the blade 10D has a length along the horizontal axis 15D of about 2.25 mm, the distal portion 14D of the blade 10D has a length of between about 0.5 mm and about 0.65 mm with a width of about 0.6 mm at the central portion 20D and then tapering to a point 36D, and the lateral edges 32D and 33D define an interior angle α of about 50 degrees. One or more of the sharpened lateral edges 21D, 22D, 32D and 33D may be beveled and may form cutting edges around the entire perimeter of the middle portion 20D and distal portion 14D of the blade 10D.

The inventors believe that the blade 10D when assembled with a handle in a knife or cutting instrument as described above may be well-suited for the formation of the side ports or incisions in the cornea which can improve the speed and efficiency of surgery and reduce trauma to the eye. Furthermore, the bend 13D in the blade 10D permits the blade 10D to be multipurpose, wherein the bend 13D has its own sharpened lateral edges that can be further advantageous for making different sized incisions in the cornea.

A sixth embodiment of a surgical instrument of the present invention is illustrated in FIG. 6, wherein only the blade 10E of the instrument is illustrated. It will be understood by one of ordinary skill in the art that the blade 10E would be assembled with a handle (such as the handle 50 in FIG. 10) to form a knife for ophthalmic surgery. The numbered features of the sixth embodiment of the blade 10E illustrated in FIG. 6 are designated generally with the suffix letter “E” and are analogous to features of the fifth embodiment of the blade 10D that share the same number (without the suffix letter “E”). The sixth illustrated embodiment of the blade 10E functions identically to the blade 10D of the fifth illustrated embodiment. The blade 10E differs in that the proximal portion 12E of the blade 10E does not possess any apertures for mounting or assembling with a handle. The blade 10E further differs in that the latera edges 21E, 22E, 32E and 33E include a bevel 38E to form cutting edges around at least the entire perimeter of the middle portion 20E and distal portion 14E of the blade 10E.

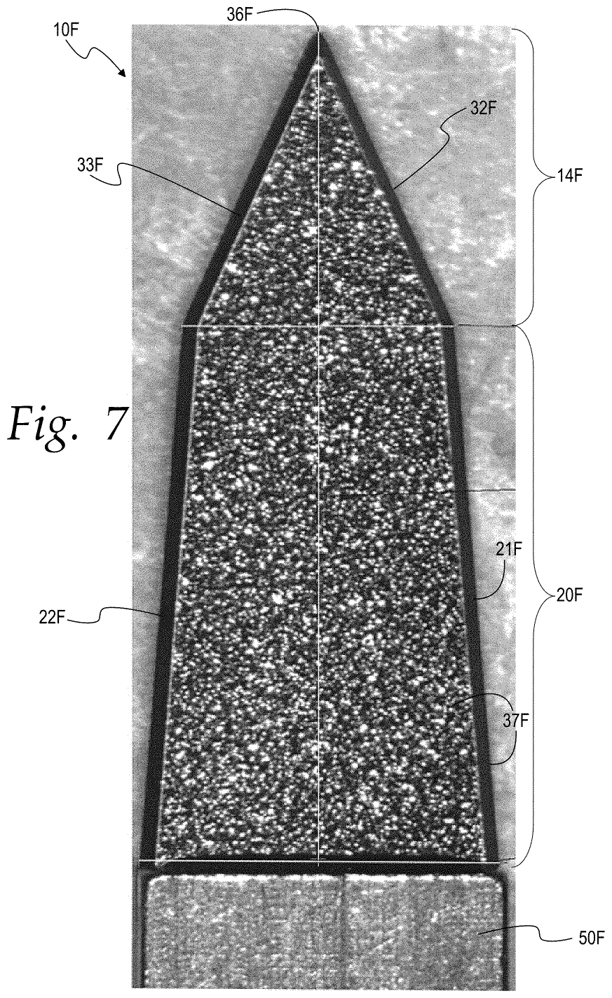

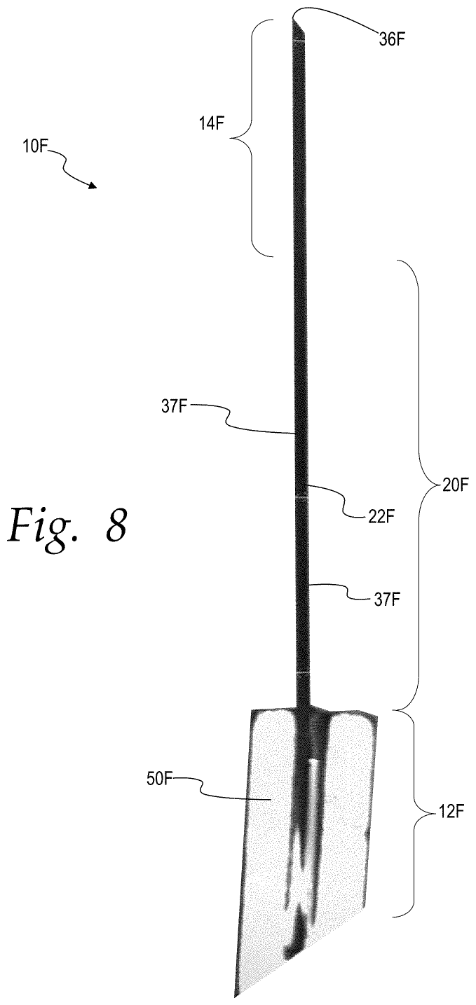

A seventh embodiment of a surgical instrument of the present invention is photographed in FIGS. 7 and 8, wherein the blade 10F of the instrument is illustrated as assembled with a handle 50F (only the distal portion of which is illustrated). It will be understood by one of ordinary skill in the art that the handle 50F may take any elongate shape sufficient for being gripped and manipulated by a user of the instrument for ophthalmic surgery. The numbered features of the seventh embodiment of the blade 10F illustrated in FIG. 6 are designated generally with the suffix letter “F” and are analogous to features of the third embodiment of the blade 10B that share the same number (without the suffix letter “F”). The seventh illustrated embodiment of the blade 10F functions identically to the blade 10B of the third illustrated embodiment, however the blade 10F is larger and more suited for making the main incision or port.

With reference to FIG. 7, it can be seen that the surfaces 37F of the blade 10F are formed from an artificial, black diamond (carbon). It is believed that the black diamond material may provide improved visibility to the user of the blade 10F penetration of the cornea while greatly reducing the cost of the blade 10F compared to natural diamond blades on the market. In one presently preferred form of the blade 10F, the combined length of the central portion 20F and the distal portion 14F along the central axis is about 5.6 mm with a width of about 2.5 mm at the wider end of the central portion 20F and a width of about 1.85 mm at the narrower end of the central portion 20F, and the sharpened lateral edges 32F and 33F define an interior angle α of about 50 degrees. A bevel extends around the sharpened lateral edges 21F, 22F, 32F and 33F to form cutting edges around the entire perimeter of the middle portion 20F and distal portion 14F of the blade 10F. In one presently preferred form of the blade 10F, the blade 10F has a nominal thickness of between 100 μm and 150 μm, and the bevel is about 30 degrees between the sides 37F (both sides visible in FIG. 8 only) of the blade 10F. The blade 10F may alternatively be formed from natural diamond, sapphire, ruby, or stainless steel. In some applications, the blade 10F may have a coating to improve wear resistance, gliding, reflectivity, etc. In one preferred form, the coating is a chemical vapor deposition “CVD” or a diamond-like deposit on the substrate of materials discussed above. The blade 10F or coating on a substrate of the blade 10F may be formed with hydrophobic and/or hydrophilic surfaces to enhance the gliding (i.e., the reduction of friction) of the blade 10F.

It will further be understood that the inventive components and cutting instruments disclosed herein may be incorporated into a larger machine or device, whereby the handle is connected to such a machine or device, and may be controlled, operated, or manipulated by such a machine or device and not necessarily by the hand of the user.

Other features and advantages will be readily apparent from the following the accompanying drawings and the appended claims.

1. A surgical blade for being combined with an elongate handle to form a cutting instrument for ophthalmic surgery, the surgical blade comprising:
a proximal portion for being attached to an elongate handle;
an operative, distal portion;
a central longitudinal axis extending between said proximal portion and said distal portion;
a central portion extending between said proximal portion and said distal portion, said central portion having a trapezoidal configuration that tapers radially inwardly toward said central longitudinal axis from said proximal portion to said distal portion, said central portion having a pair of sharpened lateral edges;
wherein said operative, distal portion includes a first tapered section defining a pair of sharpened lateral edges, said first tapered section extending from said central portion along said central longitudinal axis to a substantially straight section having a pair of sharpened lateral edges, said substantially straight section extending from said first tapered section to a second tapered section, said second tapered section having a pair of sharpened lateral edges and terminating at a tip.

2. The surgical blade in accordance with claim 1, wherein said first tapered section defines an internal angle of about 50 degrees between said pair of sharpened lateral edges.

3. The surgical blade in accordance with claim 1, wherein said second tapered section defines an internal angle of about 50 degrees between said pair of sharpened lateral edges.

4. The surgical blade in accordance with claim 1, wherein said substantially straight section defines a substantially constant width, in a radial direction relative to said central longitudinal axis between said pair of sharpened lateral edges.

5. The surgical blade in accordance with claim 1, wherein a total length of said central portion is between about 1.5 and 1.7 times greater than a total length of said distal portion, taken along said central longitudinal axis.

6. The surgical blade in accordance with claim 1, wherein said distal portion defines a total length, taken along said central longitudinal axis, and said first tapered section has a length of about 62.5% of said total length, said substantially straight section has a length of about 12.5% of said total length, and said second tapered section has a length of about 25% of said total length

7. The surgical blade in accordance with claim 1, wherein said first tapered section has a maximum width of between about 3 and 3.5 times greater than a maximum width of said second tapered section, taken in the radial direction relative to said central longitudinal axis.

8. The surgical blade in accordance with claim 1, wherein said sharpened lateral edges are beveled between top and bottom sides of said blade.

9. The surgical blade in accordance with claim 1, wherein said blade has a thickness between top and opposite bottom sides of between about 100 and 150 micrometers.

10. The surgical blade in accordance with claim 1, wherein said blade is formed from black diamond.

11. The surgical blade in accordance with claim 1, in combination with an elongate handle, wherein the combination of said blade and said handle form a surgical instrument.

12. A surgical blade for being combined with an elongate handle to form a cutting instrument for ophthalmic surgery, the surgical blade comprising:
a proximal portion for being attached to an elongate handle;
an operative, distal portion;
a central longitudinal axis extending between said proximal portion and said distal portion;
a central portion extending between said proximal portion and said distal portion, said central portion having a trapezoidal configuration that tapers radially inwardly toward said central longitudinal axis from said proximal portion to said distal portion, said central portion having a pair of sharpened lateral edges;
wherein said operative, distal portion tapers to a tip and defines a pair of sharpened lateral edges said distal portion defines an internal angle of about 50 degrees between said pair of sharpened lateral edges.

13. The surgical blade in accordance with claim 12, wherein said blade has a thickness between top and opposite bottom sides of between about 100 and 150 micrometers.

14. The surgical blade in accordance with claim 12, wherein said blade is formed from black diamond.

15. The surgical blade in accordance with claim 12, in combination with an elongate handle, wherein the combination of said blade and said handle form a surgical instrument.

16. A surgical blade for being combined with an elongate handle to form a cutting instrument for ophthalmic surgery, the surgical blade comprising:
a proximal portion for being attached to an elongate handle;
an operative, distal portion;
a central longitudinal axis defined by said proximal portion;
a bend leading toward a central portion and an operative, distal portion, said bend defining a pair of opposite internal and external sharpened edges.

17. The surgical blade in accordance with claim 16, wherein said blade has a thickness between top and opposite bottom sides of between about 100 and 150 micrometers.

18. The surgical blade in accordance with claim 16, wherein said blade is formed from black diamond.

19. The surgical blade in accordance with claim 16, in combination with an elongate handle, wherein the combination of said blade and said handle form a surgical instrument.
